(19) United States
(12) Patent Application Publication
(10) Pub. No.: US 2025/0352265 A1
(43) Pub. Date: Nov. 20, 2025

(54) MEDICAL ENDODEVICE

(71) Applicant: UNIVERSITÄT BASEL, Basel (CH)

(72) Inventors: Yukiko TOMOOKA, Basel (CH); Manuela EUGSTER, Emmen (CH); Georg RAUTER, Eiken (CH)

(21) Appl. No.: 19/120,100

(22) PCT Filed: Nov. 3, 2023
(86) PCT No.: PCT/EP2023/080676

§ 371 Date: Apr. 10, 2025

(30) Foreign Application Priority Data
Nov. 3, 2022 (EP) 22205228.4

(51) Int. Cl. A61B 18/20; A61B 18/00; A61B 34/30

(52) U.S. Cl. CPC A61B 18/201; A61B 34/30; A61B 2018/0025; A61B 2018/00565; A61B 2018/00577; A61B 2018/00601; A61B 2018/00982; A61B 2018/20553

(57) ABSTRACT

A medical endodevice (1) for an intervention inside a body cavity (54) of a body of a human or animal being (5), comprising an elongated liaising structure (2; 29), an intervention tool (7), a positioning unit (3), a decoupling structure and at least two expandable members (62). The elongated liaising structure (2; 29) has a distal end (211; 2119) arrangeable in the body cavity (54) and a proximal end arrangeable outside the body while the distal end (211; 2119) is in the body cavity (54). The intervention tool (7) is arranged to manipulate a target tissue inside the human or animal body, wherein the intervention tool (7) is arranged at the distal end (211; 2119) of the liaising structure (2; 29). The positioning unit (3) has a moving formation (32) arranged to dislocate the intervention tool (7) relative to the target tissue. The decoupling structure is arranged to decouple the positioning unit (3) once it is arranged in the body cavity (54). The at least two expandable members (62) are mounted to the positioning unit (3) and configured to fix the positioning unit (3) in the body cavity (54) when being expanded.

### TECHNICAL FIELD

The present invention relates to a medical endodevice according to the preamble of independent claim 1. Such endodevices comprising an elongated liaising structure with a distal end arrangeable in a body cavity and a proximal end arrangeable outside a body of a human or animal being while the distal end is in the body cavity, an intervention tool arranged to manipulate a target tissue inside the human or animal body, wherein the intervention tool is arranged at the distal end of the liaising structure, a positioning unit having a moving formation arranged to dislocate the intervention tool relative to the target tissue, and a decoupling structure arranged to decouple the positioning unit once it is arranged in the body cavity, can be used for interventions inside the body cavity of the body of the human or animal being.

### BACKGROUND ART

For allowing minimal invasive interventions inside a human or animal body, it is known to use devices which are forwarded through a body lumen to a target location. There, a suitable tool of the device such as a drill, a saw or the like intervenes tissue at the target location. For example, it is known to equip an endoscope with a tool, to forward the endoscope face through a body lumen and to apply the tool at a target location reached via the body lumen.

In recent years, the precision of devices like endoscopes and of the tools used with endoscopes has increased. For instance, in order to precisely controlling the endoscopes, robots are used which allow to provide sophisticated movements. Thereby, even though it was possible to have the robots to autonomously execute interventions typically operators stay involved (robot guided interventions).

With respect to the intervention tools used with endoscopes or similar devices, sophisticated instruments have been developed allowing generating precise and geometrically flexible interventions. For example, laser beams can be used for cutting or drilling hard tissue such as bone tissue or cartilage tissue. Thereby, a laser device generates laser pulses of predefined width and intensity. The pulses are directed to the tip of the endoscope, e.g. via a laser fiber, and from there beam pulses are propagated towards the target tissue. When hitting the target tissue, the laser beam pulses ablate the tissue such that holes, cuts and similar interventions can be applied to the tissue.

Today, problems in such known devices, particularly when being manually operated or controlled, arise from the fact that the precision of locating the face of the endoscope or similar device inside the body lumen typically is lower than the precision of intervention by the tool at the face of the endoscope. This might result in that the exact intervention to the target tissue by the tool is jeopardized by the lower precision of forwarding the endoscope. Therefore, there are systems in consideration which allow for increasing precision in locating the tool inside the body. For example, there are imaging procedures such as computer tomography used allowing to more precisely localizing the face of the endoscope inside the body. However, such procedures typically are cumbersome and comparably slow.

Furthermore, in operation, movements of the part of the endoscope outside the body may affect the tool inside the body during intervention. For example, a person touching or hitting the endoscope outside the body or the robot guiding the endoscope may result in movements of the tool inside the body. This affects the intervention such that its quality and precision may be lowered.

To address the downsides of common endoscopes, WO 2019/002202 A1 describes an endoscope or other medical endodevice comprising an elongated liaising structure, an intervention tool and a positioning unit. The intervention tool such as a laser emitter is arranged at the distal end of the liaising structure. The positioning unit has a moving formation and an anchoring formation configured to fix the moving formation or positioning unit to a fixing tissue inside a human or animal body. The positioning unit supports the intervention tool and can be decoupled from the liaising structure. Moreover the anchoring formation is configured to allow sufficient fixation of the positioning unit to the fixing tissue. For example, it has screw or pin means for being screwed or pinned to the fixing tissue and for a robust fixation. However, such screw, pin or other incising means may affect or harm the tissue which may be undesired. Alternatively, the anchoring formation comprises a suction mechanism for being suck to the fixing tissue. Such sucking mechanism allows for flexibly fixing and releasing the positioning unit to and from the fixing tissue without affecting the fixing tissue. However, typically sucking mechanism are complicated to set up and often are not sufficiently reliable to achieve a robust fixation.

Therefore, there is a need for a system or device allowing a precise minimal invasive intervention inside a human or animal body with minimal affection of the human or animal body.

### DISCLOSURE OF THE INVENTION

According to the invention this need is settled by a medical endodevice as it is defined by the features of independent claim 1. Preferred embodiments are subject of the dependent claims.

In particular, the invention is a medical endodevice for an intervention inside a body cavity of a body of a human or animal being. The endodevice can be suitable for a so-called minimally invasive intervention or a minimally invasive surgery. Surgery by definition is invasive and many operations require incisions of some size, particularly in open surgery. However, minimally invasive surgery involves surgical techniques that limit the size of incisions needed. Thus, whereas open surgery usually leaves comparably large wounds that are painful and take a long time to heal, minimally invasive surgery lessens wound healing time, associated pain and risk of infection. As other known instruments, the endodevice allows for being entered into the body either via an already existing opening of the body or via a comparably small cut opening towards the interior of the body such as towards a body lumen and into the body cavity.

The body cavity can be any natural or created cavity of the human or animal body. Typically a body cavity is a space or compartment, or potential space, in the body. Often, body cavities accommodate organs and other structures or substances. For example, the body cavity can be a cavity of a joint such as, specifically, a knee joint cavity. Usually body cavities are limited by their boundaries which may be embodied by specific walls or membranes or any other structure. For example, in joints the boundary of the joint body cavity can be formed by the joint capsule or other elements inside the joint capsule. In addition thereto, the body cavity may also be an artificially created space in the body of the patient. It may also be embodied as half open space such as, e.g., created by an open surgery access.

The tissue of the boundary of the body cavity also designated as fixing tissue can be a hard tissue such as the tissue of a comparably rigid structure, e.g., at least one bone, cartilage, tooth, a combination thereof or the like. The target tissue can form part of a rigid structure or it can be a tissue of another distinct rigid or soft structure. Thus, the target tissue can be the same tissue or element as the fixing tissue, or it can be a different tissue or element.

The term “endodevice” in connection with the invention relates to a device which is arranged or embodied to be introduced into the body or body lumen and to be advanced through the body or body lumen to the body cavity where the intervention is to be executed. Thereby, the term “in the body” or “inside the body” can mean any location in the human or animal body and particularly a quasi-embedded location which is not directly accessible from the outside. For example, in the body can mean in between different tissues of the body, such as in between a bone and its surrounding tissue, or inside a body lumen.

The term “body lumen” can relate to an inside space of a tubular structure in a human or animal body or to a cavity inside the human or animal body. For example, the body lumen can be a vascular vessel, such as a vein or an artery or a coronary or intracranial vessel or a heart valve, or a tract of a gastrointestinal organ such as stomach or colon, or a region of urinary collecting ducts or of renal tubes, or an interior space of joint, or a mouth or ear, or a combination thereof.

The endodevice can be or comprise a rigid or particularly a flexible endoscope, a catheter, a laparoscope, a colonoscope or a similar arrangement.

The medical endodevice comprises an elongated liaising structure, an intervention tool, a positioning unit, a decoupling structure and at least one expandable member.

The elongated liaising structure has a distal end arrangeable in the body cavity and a proximal end arrangeable outside the body while the distal end is in the body cavity. The intervention tool is arranged or configured to manipulate a target tissue inside the human or animal body and, particularly, in the body cavity, e.g., accessible via a body lumen. It is arranged at the distal end of the liaising structure.

The term “proximal” as used herein can relate to a direction towards an operator of the endodevice or a machine such as a robot controlling the endodevice. Analogously, the term “distal” can relate to a direction away from the operator or machine.

The term “manipulating the target tissue” as used in connection with the intervention tool can relate to any intervention to the target tissue such as drilling a hole, cutting, grinding, reshaping, a combination thereof or the like. It may further be adding components or structures to the target tissue such as adding an implant, additive manufacturing such as 3D printing on the target tissue, inserting a stent or the like. The manipulation of the target tissue can also cover status recoding of the target tissue such as taking a picture of the target tissue or scanning the target tissue in any manner.

The term “workspace” in connection with the intervention tool relates to a space or environment in which the intervention tool can manipulate the target tissue. Thus, it can be the operating volume which can be reached by the intervention tool.

The positioning unit has a moving formation arranged to dislocate the intervention tool relative to the target tissue. In particular, the moving formation can reposition and/or orientate the intervention tool when the positioning unit is fixed in the body cavity to allow precise incision or ablation of the target tissue.

The decoupling structure is arranged to decouple the positioning unit from the liaising structure and, particularly, once the positioning unit is arranged in the body cavity. The positioning unit can be decoupled from any guiding structure such as a tube of an endoscope, a rod or the like, and particularly from the liaising structure or a portion thereof. Thereby, the decoupling structure can be configured to decouple the positioning unit from remote or when the positioning unit is not directly accessible such as when it is positioned inside a body lumen, the body cavity or the like. Advantageously, after being decoupled, the positioning unit may still be accurately located in the body cavity such as by means of a dislocating arrangement of the positioning unit itself.

In this context, the term “decouple” is not limited to physically separating the positioning unit. Rather, it can relate to detach or uncouple the positioning unit such that it is essentially independent from movements of the component it is decoupled from. Thus, by decoupling the positioning unit, it can be arranged essentially independent from movements of the element it is decoupled from, e.g. the guiding or liaising structure. For example, if the positioning unit is coupled to a tube or rod of an endoscope such decoupling allows for making the positioning unit independent from movements of the tube or rod once it is fixed. The decoupling structure can, e.g., be embodied by a soft or flexible part. Or, in case of a flexible endoscope, decoupling can be achieved by releasing the tension of the wires or Bowden cables controlling the endoscope such that the endoscope or liaising structure can no longer move the positioning unit.

Preferably, the decoupling structure is arranged or configured to recouple the positioning unit after being decoupled. Such a recoupling allows for reconnecting the positioning unit after intervention, e.g. cutting or drilling the target tissue, such that the endodevice together with the positioning unit can conveniently be removed from or pulled out of the body after intervention. To conveniently recouple the positioning unit the decoupling structure can be configured not to completely separate the liaising structure from the positioning unit but to keep a loose or flexible connection between the two.

The at least one expandable member is mounted to the positioning unit. It is configured or designed to fix the positioning unit in the body cavity when being expanded. In particular, when being expanded, the expandable member can be pressed onto a boundary of the body cavity. Thereby, the positioning unit can securely be held or fixed in the body cavity.

For moving the positioning unit inside the body cavity, it can be equipped with a dislocation arrangement. Such dislocation arrangement can, e.g., have legs or leg-like elements allowing a walking-like moving of the positioning unit. In such embodiments, the at least one expandable member may be mounted to the dislocation arrangement.

The term “fix” as used in connection with the at least one expandable member may relate to locating the positioning unit in an essentially non-variable position or at an essentially predefined location. Fixing the positioning unit may relate to locating the positioning in a predefined relation to the fixing tissue. Like this, it can be prevented that the positioning unit is moved relative to the target tissue other than by the moving formation itself such as, e.g., by the liaising structure or any acting structure of the body such as a muscle or the like. More particularly, when being fixed, the moving formation can precisely locate and orientate the intervention member without being affected by external impacts or disturbances and without having to compensate any such impacts or disturbances. Thus, the combination of expandable member and moving formations in the positioning unit allows for an efficient and robust implementation of a highly accurate mechanism to direct the intervention member.

Thus, the at least one expandable member allows for fixing the positioning unit in an efficient and precise manner without requiring any piercing or incision of any tissue of the body and, in particular, any piercing and incision of the target tissue or a tissue neighboring the target tissue. Like this, a precise minimal invasive intervention inside a human or animal body with minimal affection of the human or animal body can be achieved.

The at least one expandable member preferably is dimensioned in accordance with the body cavity to fix the positioning unit in the body cavity when being expanded by being pressed against a boundary of the body cavity. In particular, the body cavity can be a specific body cavity such as a joint cavity and the expandable member can be dimensioned to be pressed against a boundary of the joint cavity such as a knee cavity when being expanded.

Preferably, the at least one expandable member comprises at least two expandable members. By providing two expandable members, it can be achieved that the positioning unit is securely held in the body cavity. In particular, appropriate pressing onto the boundary of the body cavity can be achieved which causes the positioning unit to be safely held.

In a preferred embodiment, two expandable members of the at least two expandable members preferably are arranged at essentially opposite sides of the positioning unit. Like this, the positioning unit can be held in between opposite walls or other boundary structures of the body cavity by pressing the expandable members to the opposite walls or other boundary structures of the body cavity.

In a preferred embodiment, two expandable members of the at least two expandable members are arranged at a single side of the positioning unit. By having the two expandable members at one single side, the positioning unit and, thus, the intervention tool can be pivoted or tilted. Like this, an orientation of the intervention tool can be adjusted.

Preferably, the at least two expandable members are individually expandable. Like this, the location and/or orientation of the positioning unit between opposite walls or other boundary structures of the body cavity, or relative to one wall or boundary structure can be efficiently set. In particular, by expanding one of the expandable members more than the other one, the positioning unit can be located non-centrally in the body cavity or tilted relative to the body cavity.

Preferably, the endodevice comprises a spacer element, wherein the spacer element and one of the at least one expandable member are arranged at essentially opposite sides of the positioning unit. By means of the spacer element, it can be achieved that a distance of the target tissue to the positioning unit is well defined. This may allow for an efficient and accurate inspection and/or manipulation of the target tissue. Such inspection and/or manipulation may involve analysis and/or palpation of the target tissue, for example using optical components such as optical coherence tomography (OCT), cameras with a fixed focal length or the like. The spacer element may comprise a spike or the like. It can be configured to be connected to the tissue.

The at least one expandable member can be embodied by any suitable expandable and collapsible structure. For example, each of the at least one expandable member can comprise a foldable element configured to unfold for expansion. Preferably, each of the at least one expandable member comprises a balloon and an inflation/deflation structure configured to inflate and deflate the associated balloon. The balloon can be made of any suitable elastic material, which is sufficiently robust to withstand the required pressure. By embodying the expandable member as balloons, a contact surface of a wall or boundary of the cavity, potentially being uneven, can efficiently be used. More specifically, uneven walls or boundaries can be levelled out by the balloon. Furthermore, such balloon allows for a precise positioning of the positioning unit when being fixed in the body cavity. Also, such balloon expandable member allows for the positioning unit being comparably compact when the balloon is deflected such that a comparably smooth and convenient insertion into the body can be achieved. Moreover, inflating and deflating the balloon allows for efficient adaptation of the position of the positioning unit inside the body cavity, as the need may be. The inflation/deflation structure can be embodied as or comprise a pump. Still further, such balloons may prevent or at least reduce harming the tissue of the body cavity.

Preferably, the medical endodevice comprises an expandable outer balloon tightly connected to the liaising structure and encasing the distal end of the liaising structure, the positioning unit and advantageously the at least one expandable member. At least a tissue contacting face of the expandable outer balloon advantageously is made of a material compliant with a tissue forming the wall of the body cavity. The expandable outer balloon can generally arrange the distal end of the liaising structure and the positioning unit at an appropriate position in the body cavity by being expanded and pressed against a wall of the body cavity. The at least one expandable member can then accurately position the positioning unit inside the expanded or inflated outer balloon and, thus, in the body cavity. Furthermore, the outer balloon can create space in the body cavity and protect the positioning unit, intervention tool and other components of the device. The outer balloon may also achieve a proper displacement of movement of the positioning unit in the body cavity. Furthermore, it can protect the tissue of the body cavity in terms of mechanical stress as well as contamination or infection. In alternative embodiments where it is less desired to protect and pre-arrange the distal end of the liaising structure, the expandable outer balloon may also be tightly connected to the positioning unit and encasing the positioning unit and the at least one expandable member.

In embodiments having additionally a spacer, the spacer can be mounted inside the outer balloon or outside the outer balloon. For example, the outer balloon may only encapsulate an upper part of the device while the spacer is mounted on a lower part of the device and is in touch with the surrounding tissue. The outer balloon can fully or only partially encapsulate the intervention tool.

The at least one expandable member can be embodied integrally with the outer balloon. Like this, the outer balloon can include the at least one expandable member. This allows to manufacture these two components together or in one processing step. In such embodiments, the at least one expandable member may be indirectly mounted to the positioning unit, i.e., via the outer balloon. Further, be it integral with the outer balloon or not, the at least one expandable member can be located or positioned at an outside of the outer balloon. Thereby, the outer balloon may be arranged or designed to not encase the at least one expandable member.

Preferably, the medical endodevice comprises a laser arrangement, wherein the intervention tool is a laser beam propagating structure of the laser arrangement configured to propagate a laser beam. The laser beam can particularly be suitable to ablate the target tissue. Advantageously, it is a pulsed laser beam.

Such laser devices are becoming increasingly popular since they allow ablating bone or other hard tissue in a very precise and gentle manner without requiring mechanical interaction forces. As such lasers allow for providing a comparably high precision, the endodevice and particularly its positioning unit can be specifically advantageous.

The expandable outer balloon preferably is at least partially transparent to the laser beam propagated by the laser beam propagating structure of the laser arrangement. In particular, the expandable outer balloon can have a window or saphire window transparent to the laser beam or can be completely transparent to the laser beam. Such outer balloon allows for an efficient protected laser provision.

Further, the outer balloon may be provided with an opening. Such opening may allow accessing the exterior of the balloon. For example, when the endodevice is equipped with a gripper, said gripper may access any tissue or structure outside the outer balloon via the opening. The opening can also be provided as transparent portion through which the laser beam may be directed.

The laser beam propagating structure of the laser arrangement preferably comprises an adjustable optics arranged to direct the laser beam in various directions. The optics can particularly comprise at least one mirror. Such an optics allows for precisely directing the laser beam such that a broad variety of intervention geometries can be implemented. In particular, the optics can also be adjusted for an ablation orthogonal to the bone or to a surface thereof.

In addition to the intervention tool, the endodevice can also be equipped with one or more further tools or instruments such as a gripper, a camera, a suction module or the like. Such further tools can be coupled to or directed by the moving formation such that they can benefit from the advantageous operability provided by the moving and fixing formations of the positioning unit.

Preferably, the laser arrangement comprises an optical fiber connectable to a laser source, the optical fiber has a distal end from which the laser beam is ejectable, and the laser beam propagating structure of the laser arrangement comprises the distal end of the optical fiber of the laser arrangement. Such embodiment of the laser arrangement allows for efficiently implementing the intervention tool propagating a laser beam at comparably little required space.

The laser arrangement can comprise further components which end or are located in or near the positioning unit. For example, the laser arrangement can have a suction device for removing debris of the tissue when being ablated by the laser, a camera for observing the laser ablation, a depth measuring device for identifying how deep the ablation goes into the tissue, an optical coherence tomography device for providing an overview of the ablation process, similar auxiliary devices or combinations thereof.

Preferably, the moving formation of the positioning unit has a first rail, a first slide and a first arm, wherein the first slide is mounted to the first rail such that it is movable along the first rail, and the first arm is at one end region rotatably mounted to the first slide. For example, like this an axial movement, a lateral movement and a pivoting, e.g. in the same plane, may be achieved. Such three degrees of freedom may be appropriate for many applications and intervention tools such as laser systems.

The moving formation of the positioning unit can have a second rail, a second slide and a second arm, wherein the second slide is mounted to the second rail such that it is movable along the second rail and the second arm is at one end region rotatably mounted to the second slide. The first rail of the moving formation of the positioning unit and the second rail of the moving formation of the positioning unit advantageously are parallel to each other.

The moving formation of the positioning unit preferably has a further first rail, a further first slide and a further first arm, wherein the further first slide is mounted to the further first rail such that it is movable along the further first rail, and the further first arm is at one end region rotatably mounted to the further first slide and. Further, the moving formation of the positioning unit may have a further second rail, a further second slide and a further second arm, wherein the further second slide is mounted to the further second rail such that it is movable along the further second rail and the further second arm is at one end region rotatably mounted to the further second slide. The further first rail of the moving formation of the positioning unit and the further second rail of the moving formation of the positioning unit advantageously are parallel to each other.

Preferably, the positioning unit comprises a sensor arranged to localize the positioning unit. Such a sensor allows for preventing imaging such as computer tomography (CT) to be mandatory. Also, it allows for setting up a closed loop system automatically correcting any erroneous position changes of the positioning unit, e.g., induced by the body or by manipulation of the portions of the medical endodevice outside the body. Thereby, the sensor preferably is an optical sensor or any other suitable sensor such as an accelerometer, a gyroscope or any combination thereof. Such sensors can be sufficiently precise and fast.

Preferably, the medical endodevice comprises a robot arrangement connected to the intervention tool and the positioning unit via the liaising structure. Such a robot allows for a (semi-)automated controlling of the intervention tool and the positioning unit.

Advantageously, the moving formation of the positioning unit is arranged or configured to dislocate the intervention tool relative to the target tissue in three to five or six degrees of freedom. Such arrangement might allow for sufficient, precise and fast positioning of the intervention tool. In other embodiments, the moving formation can be arranged or configured to dislocate the intervention tool in more degrees of freedom. For example, movements can comprise lateral movements, back and forth movements, tilting movements, rotations about the longitudinal axis of the endodevice or the like. Such dislocation in any suitable number of degrees of freedom can be implemented by the moving formation described above. In particularly, the moving formation can be embodied with slides coupled to rails. Additionally or alternatively, the legs of the fixing formation can be length adjustable. Further, additionally or alternatively, one or more joint or pivoting structure can be implemented allowing a rotation or tilting movement. Thus, the moving formation can be equipped with any suitable element to achieve the desired degrees of freedom of motion. The moving formation can be embodied as or comprise a robot or robot like structure. For example, it can comprise a parallel robot or a similar device.

Advantageously, the liaising structure of the endodevice comprises a tube ending at its distal end. The tube can be embodied to receive intervention tools or other instruments in operation. For example, in operation of the endodevice a drilling tool can be forwarded through the tube. Furthermore, the tube can end in the positioning unit such that variable tools or instruments can be precisely positioned by the positioning unit.

For navigating the positioning unit and the intervention tool, the medical endodevice can be equipped with any suitable means such as a pneumatic or hydraulic formation, a shape memory alloy, an embedded motor, a microelectromechanical system (MEMS), or a combination thereof or the like. However, in a preferred embodiment, the liaising structure comprises at least one navigation wire fixed to the moving formation of the positioning unit. The term “wire” in this context can relate to any cable, rope, filament, fiber, yarn, cord, string, Bowden cable, torsion spring or the like of any suitable material. A suitable material could be biocompatible and should be robust enough for allowing controlling the positioning unit via the at least on wire. Such a wire allows for manipulating the positioning unit and in particular to activate the anchoring formation once the positioning unit is at an appropriate location as well as to operate the moving formation for adjusting the location of the intervention tool.

Thereby, the at least one navigation wire of the liaising structure preferably is mounted to the first slide of the moving formation of the positioning unit. Like this, the first slide can be moved along the first rail by manipulating such as pulling and pushing the wire. Analogously, further wires of the liaising structure can be mounted to the second slide, the further first slide and the further second slide. Also, the slides can be connected to springs which push or pull the slides away from the wires. Like this, the slides can be manipulated by pulling and releasing the wires. Also, rotational springs actuating spindles in the first or other slides can be provided for moving or advancing the slides.

Another aspect of the present disclosure is a medical endo-apparatus for an intervention inside a body cavity of a body of a human or animal being. The medical endo-apparatus comprises an elongated liaising structure having a distal end arrangeable in the body cavity and a proximal end arrangeable outside the body while the distal end is in the body cavity, an intervention tool arranged to manipulate a target tissue inside the human or animal body, wherein the intervention tool is arranged at the distal end of the liaising structure, a positioning unit having a moving formation arranged to dislocate the intervention tool relative to the target tissue, and a decoupling structure arranged to decouple the positioning unit once it is arranged in the body cavity. The endo-apparatus further comprises an expandable outer balloon tightly connected to the liaising structure and at least partially encasing the distal end of the liaising structure and the positioning unit.

Such endo-apparatus allows for providing sterility of the workspace where the intervention tool manipulates the target tissue inside the human or animal body. In particular, such a balloon may protect the tissue of the body cavity as well as the intervention tool and components adjacent to it. Like this, a safe and efficient inspection and/or manipulation of the target tissue can be achieved. Such inspection and/or manipulation may involve analysis and/or palpation of the target tissue, for example using optical components such as optical coherence tomography (OCT), cameras with a fixed focal length or the like.

As described above in connection with the endodevice according to the invention, at least a tissue contacting face of the expandable outer balloon advantageously is made of a material compliant with a tissue forming the wall of the body cavity. The expandable outer balloon can generally arrange the distal end of the liaising structure and the positioning unit at an appropriate position in the body cavity by being expanded and pressed against a wall of the body cavity. The at least one expandable member can then accurately position the positioning unit inside the expanded or inflated outer balloon and, thus, in the body cavity. Furthermore, the outer balloon can create space in the body cavity and protect the positioning unit, intervention tool and other components of the device. The outer balloon may also achieve a proper displacement of movement of the positioning unit in the body cavity. Furthermore, it can protect the tissue of the body cavity in terms of mechanical stress as well as contamination or infection. In alternative embodiments where it is less desired to protect and pre-arrange the distal end of the liaising structure, the expandable outer balloon may also be tightly connected to the positioning unit and encasing the positioning unit and the at least one expandable member. In embodiments having additionally a spacer, the spacer can be mounted inside the outer balloon or outside the outer balloon. For example, the outer balloon may only encapsulate an upper part of the device while the spacer is mounted on a lower part of the device and is in touch with the surrounding tissue. The outer balloon can fully or only partially encapsulate the intervention tool.

The endo-apparatus can be equipped with the features described above in connection with the endodevice according to the invention such that the effects and benefits described above in connection with said features are achieved. In the following such advantageous features of the endo-apparatus are listed.

Preferably, at least a tissue contacting face of the expandable outer balloon is made of a material compliant with a tissue forming the wall of the body cavity.

Preferably, the endo-apparatus comprises at least one expandable member mounted to the positioning unit and configured to fix the positioning unit in the body cavity when being expanded, wherein the outer balloon encases the at least one expandable member.

The at least one expandable member can be embodied integrally with the outer balloon. Like this, the outer balloon can include the at least one expandable member. This allows to manufacture these two components together or in one processing step. In such embodiments, the at least one expandable member may be indirectly mounted to the positioning unit, i.e., via the outer balloon. Further, be it integral with the outer balloon or not, the at least one expandable member can be located or positioned at an outside of the outer balloon. Thereby, the outer balloon may be arranged or designed to not encase the at least one expandable member.

The at least one expandable member preferably comprises at least two expandable members.

Two expandable members of the at least two expandable members preferably are arranged at essentially opposite sides of the positioning unit.

Two expandable members of the at least two expandable members preferably are arranged at a single side of the positioning unit.

The at least two expandable members preferably are individually expandable.

Preferably, medical endo-apparatus comprises a spacer element, wherein the spacer element and one of the at least one expandable member are arranged at essentially opposite sides of the positioning unit.

Preferably, each of the at least one expandable member comprises a balloon and an inflation/deflation structure configured to inflate and deflate the associated balloon.

Preferably, the endo-apparatus comprises a laser arrangement, wherein the intervention tool is a laser beam propagating structure of the laser arrangement configured to propagate a laser beam. Thereby, the expandable outer balloon preferably is at least partially transparent to the laser beam propagated by the laser beam propagating structure of the laser arrangement. The laser beam propagating structure of the laser arrangement preferably comprises an adjustable optics arranged to direct the laser beam in various directions.

Preferably, the laser arrangement comprises an optical fiber connectable to a laser source, the optical fiber has a distal end from which the laser beam is ejectable, and the laser beam propagating structure of the laser arrangement comprises the distal end of the optical fiber of the laser arrangement.

Preferably, the moving formation of the positioning unit has a first rail, a first slide and a first arm, wherein the first slide is mounted to the first rail such that it is movable along the first rail, and the first arm is at one end region rotatably mounted to the first slide. Thereby, the moving formation of the positioning unit preferably has a further first rail, a further first slide and a further first arm, wherein the further first slide is mounted to the further first rail such that it is movable along the further first rail, and the further first arm is at one end region rotatably mounted to the further first slide.

Preferably, the at least one expandable member is dimensioned in accordance with the body cavity to fix the positioning unit in the body cavity when being expanded by being pressed against a boundary of the body cavity.

Preferably, the medical endo-apparatus comprises a robot arrangement connected to the intervention tool and the positioning unit via the liaising structure.

Preferably, the decoupling structure is arranged to recouple the positioning unit after being decoupled.

### BRIEF DESCRIPTION OF THE DRAWINGS

The medical endodevice according to the invention and the endo-apparatus as described above are described in more detail herein below by way of exemplary embodiments and with reference to the attached drawings, in which:

FIG. 1 shows an overview of a first embodiment of a medical endodevice according to the invention or medical endo-apparatus in use, wherein a section comprising a positioning unit is additionally shown in an enlarged presentation;

FIG. 2 shows a schematic view of the positioning unit of the medical endodevice or medical endo-apparatus of FIG. 1 fixed in a body cavity;

FIG. 3 shows a schematic view of a positioning unit of a second embodiment of a medical endodevice according to the invention or medical endo-apparatus fixed in a body cavity; and

FIG. 4 shows a schematic view of a positioning unit of a third embodiment of a medical endodevice according to the invention or medical endo-apparatus.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented, and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

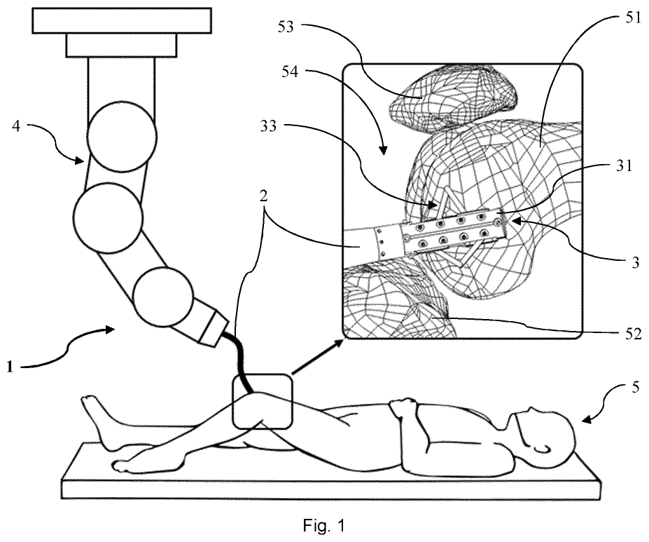

FIG. 1 shows an overview of a first embodiment of a medical endodevice 1 according to the invention or medical endo-apparatus 1 in operation. In the following the term endo-device covers endo-device and endo-apparatus. The endodevice 1 comprises an endoscope 2 as liaising structure equipped with a positioning unit 3 at its distal end and an arm robot 4 guiding and controlling the endoscope 2.

The endodevice 1 is used for an intervention in a knee of a patient 5. The endodevice 1 comprises an arm robot 4 and the endoscope 2 with a positioning unit 3. The endoscope 2 extends into an interior space 54 of the knee as body cavity via an opening cut. The interior space 54 has a boundary formed by a femur 51, a tibia 52, a patella 53 and other structures of the patient 5.

The positioning unit 3 has a base body 31 and is equipped with a dislocating arrangement 33 for moving the base body 31 in the interior space 54.

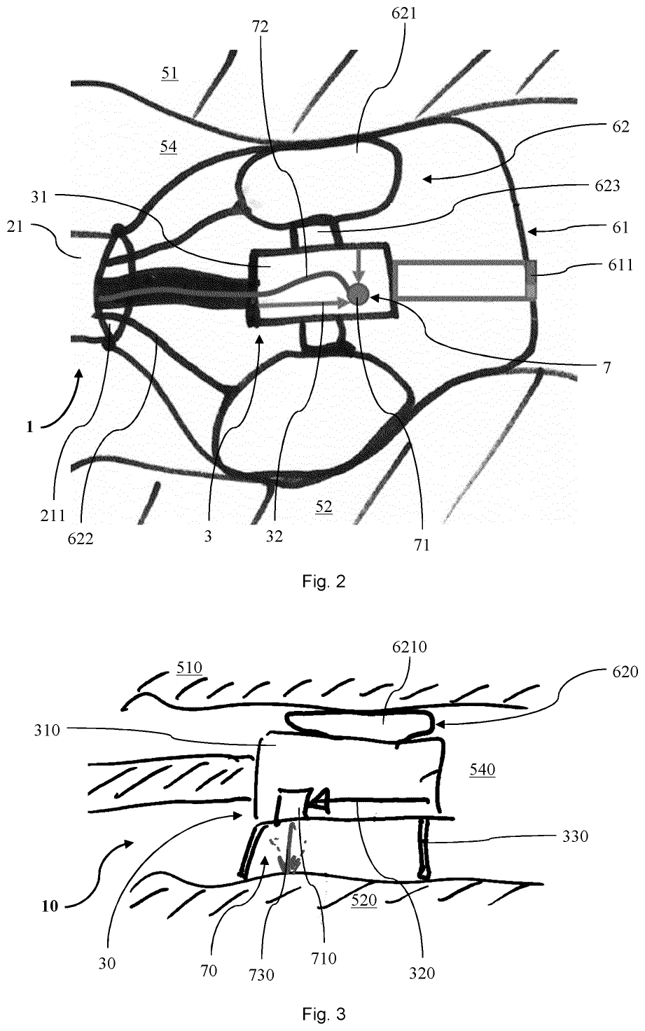

In FIG. 2 the endoscope 2 introduced in the interior space 54 is shown in more detail. The endoscope 2 has a flexible tube 21 with a distal end 211 located in the interior space 54. Distally of the tube 21 the positioning unit 3 is arranged and fixed to a boundary of the interior space 54.

The endodevice 1 comprises a laser arrangement 7 having an optical fiber 72 and a mirror 71 as beam propagating structure and intervention tool. The optical fiber 72 extends through a single channel of the tube 21 of the endoscope 2 to the mirror 71, which is arranged in the positioning unit 3. More specifically, the positioning unit 3 has a moving formation 32 for translating the mirror 71 along three axes. More specifically, the moving formation 32 has rails and a slide for providing the respective movement. in addition to being relocated by means of the moving formation 32, the mirror 71 can be tilted in and relative to the positioning unit 3 such that a pulsed laser beam emitted via the optical fiber 72 and the mirror 71 can precisely be targeted. Like this, the laser beam can be directed as desired on a target tissue to ablate the target tissue.

The endodevice 1 further has an outer balloon 61 which is tightly connected to distal end 211 of the tube 21. A distal opening of the tube 21 as well as the positioning unit 3 and all components extending between the tube 21 and the positioning unit 3 are located inside the outer balloon 61. The outer balloon 61 is inflated such that it contacts the boundary of the interior space 54. Like this, the tube 21 and the positioning unit 3 are stabilized relative to the boundary of the interior space 54 and the positioning unit 3 is protected. The outer balloon 61 is equipped with a window 611 which is transparent to the laser beam. Like this, the laser beam can be provided through the window 611 onto the target tissue to be ablated.

For fixing the positioning unit 3 once arranged at a suitable or appropriate location in the interior space 54, the positioning unit 3 is equipped with two opposite expandable members 62. Each expandable member 62 has a balloon housing 623, a balloon 621 and an inflation/deflation pipe 622 extending through the single channel of the tube (21). For expanding the balloons 621 they are selectively inflated via the respective inflation/deflation pipe 622 until they are pressed against the boundary of the interior space 54. By selectively inflating the balloons 621 a location of the positioning unit 3 inside the interior space 54 can precisely be determined. The flexibility of the balloons 621 allows to level the uneven boundary. In the fixed position shown in FIG. 2, the laser beam can precisely be provided on the target tissue.

For unfixing the positioning unit 3 in order to relocate it or withdraw it out of the body, the balloons 621 are deflated by withdrawing the air via the inflation/deflation pipes 622. Like this, the balloons 621 collapse and are arranged inside the balloon housings 623.

FIG. 3 shows a schematic illustration of some components of a second embodiment of a medical endodevice 10 according to the invention or endo-apparatus 10 in operation. The endodevice 10 comprises an endoscope as liaising structure equipped with a positioning unit 30. In FIG. 3 the positioning unit 30 is introduced in a body cavity 540 formed by a boundary having a first wall 510 and an opposite second wall 520.

The endodevice 10 comprises a laser arrangement 70 having an optical fiber and a mirror 710 as beam propagating structure and intervention tool. The mirror 710 is arranged in the positioning unit 30 and can be relocated and tilted in and relative to the positioning unit 30 such that a pulsed laser beam 730 emitted via the mirror 710 can precisely be targeted. Like this, the laser beam 730 can be directed as desired on the second wall 520 as target tissue to be ablated.

The positioning unit 30 has a base body 310 and is equipped with a stand 330 for placing the base body 310 onto the second wall 520 of the body cavity 540 as spacer.

For fixing the positioning unit 30 once placed on the second wall 520 at a suitable or appropriate location, the positioning unit 30 is equipped with one expandable member 620. The expandable member 620 has a balloon 6210 and an inflation/deflation pipe. When inflating and expanding the balloon 6210 it is pressed against the first wall 510 of the body cavity 540. Like this, the positioning unit 30 and, specifically, its stand 330 is pushed against the second wall 520. Like this the positioning unit is solidly fixed in the body cavity 540 at the appropriate location. In the fixed position shown in FIG. 3, the laser beam can precisely be provided on the second wall as target tissue.

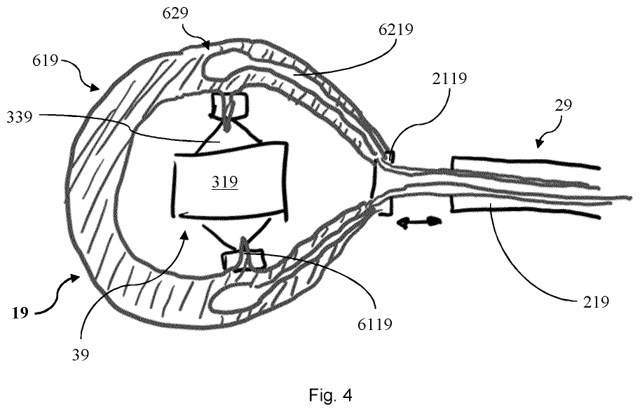

In FIG. 4 a schematic illustration of some components of a third embodiment of a medical endodevice 19 according to the invention or endo-apparatus 19 is shown. The endodevice 19 comprises an endoscope 29 as liaising structure having a tube 219 with a distal end portion 2119, and a positioning unit 39. The endodevice 19 comprises a laser arrangement as beam propagating structure and intervention tool, some components of which being arranged in the positioning unit 39.

The positioning unit 39 has a base body 319 and is equipped with two spacers 339 at opposite sides. The distal end portion 2119 is configured to be decoupled from and coupled to the tube 219.

The endodevice 19 further comprises an outer balloon 619 tightly mounted to the distal end portion 2119 and encasing the positioning unit 39 and the spacers 339. The outer balloon 619 is equipped with two opposite cavities 6219 as expandable members 629, and two connector portions 6119.

Each cavity 6219 has a chamber portion and a pipe portion. The chamber portion can be inflated and deflated by a fluid being provided through the pipe portion. Like this, the cavities 6219 can be expanded or collapsed. The outer balloon 619 is fixed to the spacers 339 by means of the connector portions 6119 such that one of the cavities 6219 is aligned and positioned relative to one of the spacers 339.

This description and the accompanying drawings that illustrate aspects and embodiments of the present invention should not be taken as limiting the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. Also, the present disclosure covers intermediate generalisations of features or groups of features of the embodiments described and shown in the figures. I.e., specific features or groups of features as disclosed in the figures and the associated sections of the description may be combined with the more general embodiments of the invention disclosed in connection with the description of the invention. In particular, such specific features or groups of features may be provided in the more general embodiments of the invention in isolation from further specific features shown in the figures. For example, one or both cavities of the outer balloon shown in FIG. 4 may be embodied as expandable member in the outer balloon according to the invention without any other feature or with some of the other features shown in FIG. 4. It is understood that those skilled in the art are able to incorporate specific features from the description of the figures into the embodiments of the description of the invention.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. A medical endodevice for an intervention inside a body cavity of a body of a human or animal being, comprising
an elongated liaising structure having a distal end arrangeable in the body cavity and a proximal end arrangeable outside the body while the distal end is in the body cavity,
an intervention tool arranged to manipulate a target tissue inside the human or animal body, wherein the intervention tool is arranged at the distal end of the liaising structure,
a positioning unit having a moving formation arranged to dislocate the intervention tool relative to the target tissue,
a decoupling structure arranged to decouple the positioning unit once it is arranged in the body cavity, and
at least one expandable member mounted to the positioning unit and being configured to fix the positioning unit in the body cavity when being expanded.

2. The medical endodevice of claim 1, wherein the at least one expandable member comprises at least two expandable members.

3. The medical endodevice of claim 2, wherein two expandable members of the at least two expandable members are arranged at essentially opposite sides of the positioning unit.

4. The medical endodevice of claim 2, wherein two expandable members of the at least two expandable members are arranged at a single side of the positioning unit.

5. The medical endodevice of claim 2, wherein the at least two expandable members are individually expandable.

6. The medical endodevice of claim 1, further comprising a spacer element, wherein the spacer element and one of the at least one expandable member are arranged at essentially opposite sides of the positioning unit.

7. The medical endodevice of claim 1, wherein each of the at least one expandable member comprises a balloon and an inflation/deflation structure configured to inflate and deflate the associated balloon.

8. The medical endodevice of claim 1, comprising an expandable outer balloon-tightly connected to the liaising structure and encasing the distal end of the liaising structure, the positioning unit.

9. The medical endodevice of claim 8, wherein at least a tissue contacting face of the expandable outer balloon is made of a material compliant with a tissue forming the wall of the body cavity.

10. The medical endodevice of claim 1, further comprising a laser arrangement, wherein the intervention tool is a laser beam propagating structure of the laser arrangement configured to propagate a laser beam.

11. The medical endodevice of claim 10, further comprising an expandable outer balloon tightly connected to the liaising structure and encasing the distal end of the liaising structure and the positioning unit, wherein the expandable outer balloon is at least partially transparent to the laser beam propagated by the laser beam propagating structure of the laser arrangement.

12. The medical endodevice of claim 10, wherein the laser beam propagating structure of the laser arrangement comprises an adjustable optics arranged to direct the laser beam in various directions.

13. The medical endodevice of claim 10, wherein
the laser arrangement comprises an optical fiber connectable to a laser source,
the optical fiber has a distal end from which the laser beam is ejectable, and
the laser beam propagating structure of the laser arrangementcomprises the distal end of the optical fiber of the laser arrangement.

14. The medical endodevice of claim 1, wherein the moving formation of the positioning unit has a first rail, a first slide and a first arm, wherein the first slide is mounted to the first rail such that it is movable along the first rail, and the first arm is at one end region rotatably mounted to the first slide.

15. The medical endodevice of claim 14, wherein the moving formation of the positioning unit has a further first rail, a further first slide and a further first arm, wherein the further first slide is mounted to the further first rail such that it is movable along the further first rail, and the further first arm is at one end region rotatably mounted to the further first slide.

16. The medical endodevice of claim 1, wherein the at least one expandable member is dimensioned in accordance with the body cavity to fix the positioning unit in the body cavity when being expanded by being pressed against a boundary of the body cavity.

17. The medical endodevice of claim 1, comprising a robot arrangement connected to the intervention tool and the positioning unitvia the liaising structure.

18. The medical endodevice of claim 1, wherein the decoupling structure is arranged to recouple the positioning unitafter being decoupled.

19. The medical endodevice of claim 8, wherein the expandable outer balloon encases the at least one expandable member.
